(19) United States
(12) Patent Application Publication
(10) Pub. No.: US 2013/0089602 A1
(43) Pub. Date: Apr. 11, 2013

(54) ENCAPSULATED CHELATOR

(71) Applicants: Dylan J. Boday, Tucson, AZ (US); Joseph Kuczynski, Rochester, MN (US); Robert E. Meyer, III, Rochester, MN (US)

(72) Inventors: Dylan J. Boday, Tucson, AZ (US); Joseph Kuczynski, Rochester, MN (US); Robert E. Meyer, III, Rochester, MN (US)

(73) Assignee: INTERNATIONAL BUSINESS MACHINES CORPORATION, Armonk, NY (US)

(21) Appl. No.: 13/269,764

(22) Filed: Oct. 10, 2011

(51) Int. Cl. A61K 9/48; A61K 31/195

(52) U.S. Cl. 424/451; 514/566; 514/567; 514/561

(57) ABSTRACT

An enhanced chelator includes a chelating agent and a volatile material encapsulated in a biologically benign microcapsule. The enhanced chelator possesses significantly improved shelf-life in aqueous biological buffer solutions because the chelating agent is encapsulated in the microcapsule and, therefore, separated from solution components with which the chelating agent would react. The enhanced chelator is activated at a predetermined elevated temperature defined by the boiling point of the volatile material. At this predetermined elevated temperature, the volatile material exerts a vapor pressure sufficient to rupture the microcapsule and thereby release the chelating agent from the microcapsule. In one embodiment, a manganese chelator such as ethylene glycol tetraacetic acid (EGTA) is solubilized in ethanol and encapsulated in a poly(lactic-co-glycolide) (PLGA) microsphere. Upon heating to 80° C., ethanol boils within the PLGA microsphere and undergoes several orders of magnitude volume change, thereby rupturing the PLGA microsphere and releasing EGTA.

### BACKGROUND OF THE INVENTION

1. Field of Invention

The present invention relates in general to the field of chelators. More particularly, the present invention relates to encapsulating a chelating agent and a volatile material in a biologically benign microcapsule.

2. Background Art

Chelation therapy is the administration of a chelating agent (also referred to as a “chelator”) to remove heavy metals from the body. For example, calcium-disodium ethylenediaminetetraacetic acid (EDTA) is approved by the U.S. Food and Drug Administration (FDA) for treating lead poisoning and heavy metal toxicity. The chelating agent may be administered intravenously, intramuscularly, or orally, depending on the agent and the type of poisoning.

Chronic exposure to excessive levels of manganese (Mn) can lead to manganese poisoning or manganism, a neurological disease with symptoms resembling those of idiopathic Parkinson's disease. A conventional treatment for manganism is chelation therapy using EDTA. Accumulation of manganese also has been associated with Alzheimer type II astrocytic changes. Studies indicate that manganese is highly accumulated in astrocytes.

While EDTA is an effective chelator for treating manganese poisoning or manganism, it is not a manganese-specific chelating agent. Manganese chelators, which have a significantly higher affinity for Mn²⁺ than other divalent metal ions (e.g., Mg²⁺), are well known in the art. Conventional manganese chelators include ethylene glycol tetraacetic acid (EGTA), para-aminosalicylic acid (PAS), 1,2-cyclohexylenedinitrilotetraacetic acid (CDTA), nitrilotriacetic acid (NAS), and diethylenetriaminepentaacetic acid (DTPA).

In the context of chelation therapy, conventional chelators (including conventional manganese chelators and other conventional metal-specific chelating agents) are typically placed in aqueous biological buffer solutions to be administered to patients. Conventional chelators (including conventional manganese chelators and other conventional metal-specific chelating agents) are utilized in aqueous biological buffer solutions in other contexts, such as for the purpose of performing an assay. Unfortunately, conventional chelators (including conventional manganese chelators and other conventional metal-specific chelating agents) are not stable for long periods of time (e.g., at least 6 weeks) in aqueous biological buffer solutions. This instability results from the conventional chelators reacting with components in aqueous biological buffer solutions, such as Tris buffer {Tris-(hydroxymethyl)-aminomethane}, KCl, divalent metal ions, nucleotides, proteins, etc.

### SUMMARY OF THE INVENTION

Some embodiments of the invention provide an enhanced chelator that is stable in aqueous biological buffer solutions for long periods of time.

According to some embodiments of the present invention, an enhanced chelator includes a chelating agent and a volatile material encapsulated in a biologically benign microcapsule. The enhanced chelator possesses significantly improved shelf-life in aqueous biological buffer solutions because the chelating agent is encapsulated in the microcapsule and, therefore, separated from solution components with which the chelating agent would react. The enhanced chelator is activated at a predetermined elevated temperature defined by the boiling point of the volatile material. At this predetermined elevated temperature, the volatile material exerts a vapor pressure sufficient to rupture the microcapsule and thereby release the chelating agent from the microcapsule. In one embodiment, a manganese chelator such as ethylene glycol tetraacetic acid (EGTA) is solubilized in ethanol and encapsulated in a poly(lactic-co-glycolide) (PLGA) microsphere. Upon heating to 80° C., ethanol boils within the PLGA microsphere and undergoes several orders of magnitude volume change, thereby rupturing the PLGA microsphere and releasing EGTA.

The foregoing and other features and advantages of the present invention will be apparent from the following more particular description of embodiments of the present invention, as illustrated in the accompanying drawings.

### BRIEF DESCRIPTION OF THE DRAWINGS

The preferred exemplary embodiments of the present invention will hereinafter be described in conjunction with the appended drawings, where like designations denote like elements.

FIG. 1 is a sectional view of an exemplary enhanced chelator that includes a chelating agent and a volatile material encapsulated in a biologically benign microcapsule in accordance with some embodiments of the present invention.

FIG. 2 is a flow diagram illustrating an exemplary method of fabricating an enhanced chelator in accordance with some embodiments of the present invention.

FIG. 3 is a flow diagram illustrating an exemplary method of activating an enhanced chelator in accordance with some embodiments of the present invention.

### DETAILED DESCRIPTION OF THE PREFERRED EMBODIMENTS

In accordance with some embodiments of the present invention, an enhanced chelator includes a chelating agent and a volatile material encapsulated in a biologically benign microcapsule. The enhanced chelator possesses significantly improved shelf-life in aqueous biological buffer solutions because the chelating agent is encapsulated in the microcapsule and, therefore, separated from solution components with which the chelating agent would react. The enhanced chelator is activated at a predetermined elevated temperature defined by the boiling point of the volatile material. At this predetermined elevated temperature, the volatile material exerts a vapor pressure sufficient to rupture the microcapsule and thereby release the chelating agent from the microcapsule. In one embodiment, a manganese chelator such as ethylene glycol tetraacetic acid (EGTA) is solubilized in ethanol and encapsulated in a poly(lactic-co-glycolide) (PLGA) microsphere. Upon heating to 80° C., ethanol boils within the PLGA microsphere and undergoes several orders of magnitude volume change, thereby rupturing the PLGA microsphere and releasing EGTA.

An enhanced chelator in accordance with the present invention may be utilized in many different applications. In the context of chelation therapy, an enhanced chelator in accordance with some embodiments of the present invention may be placed in an aqueous biological buffer solution to be administered to a patient. Also, an enhanced chelator in accordance with some embodiments of the present invention may be utilized in an aqueous biological buffer solution in other contexts, such as for the purpose of performing an assay. Unlike conventional chelators, the enhanced chelator will not react with components in aqueous biological buffer solutions, such as Tris buffer {Tris-(hydroxymethyl)-aminomethane}, KCl, divalent metal ions such as Mg²⁺, nucleotides, proteins, etc. Because the chelating agent is encapsulated in the microcapsule away from solution components with which the chelating agent would react, the enhanced chelator possesses significantly improved shelf-life even when stored in an aqueous biological buffer solution.

An enhanced chelator in accordance with some of the embodiments of the present invention may be used as an analytical tool (e.g., for the purpose of performing an assay), either in an aqueous biological buffer solution or some other solution. Also, an enhanced chelator in accordance with some embodiments of the present invention may be used for the purposes of recovery, purification and/or concentration.

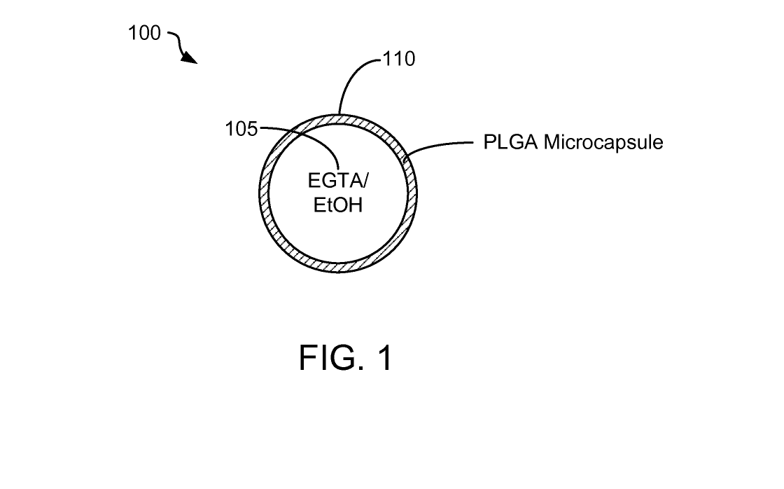

FIG. 1 is a sectional view of an exemplary enhanced chelator 100 that includes a chelating agent/volatile material core solution 105 (e.g., EGTA solubilized in ethanol, as illustrated in the exemplary embodiment of FIG. 1) encapsulated in a biologically benign microcapsule 110 (e.g., a PLGA microcapsule, as illustrated in the exemplary embodiment of FIG. 1) in accordance with some embodiments of the present invention.

In general, any suitable conventional chelating agent may be utilized in the core solution 105. Preferably, the core solution 105 includes at least one manganese chelator. Manganese chelators, which have a significantly higher affinity for Mn²⁺ than other divalent metal ions (e.g., Mg²⁺), are well known in the art. Conventional manganese chelators that are suitable for use in the core solution 105 include, but are not limited to, ethylene glycol tetraacetic acid (EGTA), para-aminosalicylic acid (PAS), 1,2-cyclohexylenedinitrilotetraacetic acid (CDTA), nitrilotriacetic acid (NAS), diethylenetriaminepentaacetic acid (DTPA), and combinations thereof. The use of EGTA (as the “chelating agent”) in the exemplary embodiment of FIG. 1 is for purposes of illustration and not limitation.

The volatile material utilized in the core solution 105 is preferably selected based on a number of criteria. First, a suitable volatile material vaporizes at a predetermined elevated temperature appropriate for intended application. For example, the boiling point of the volatile material is preferably substantially above the storage temperature of the enhanced chelator to avoid inadvertent activation of the enhanced chelator. Second, a suitable volatile material does not react with the chelating agent. That is, the chelating agent preferably remains stable in the core solution 105 during the shelf-life of the enhanced chelator. Third, a suitable volatile material is biologically benign. For example, when the enhanced chelator 100 is intended to be administered to a human patient, the volatile material must be nontoxic to humans. Fourth, a suitable volatile material does not unacceptably interfere with the encapsulation process selected to encapsulate the core solution 105. The use of ethanol (as the “volatile material”) in the exemplary embodiment of FIG. 1 is for purposes of illustration and not limitation.

In the core solution 105, the chelating agent may be solubilized in the volatile material with the aid of one or more surfactants, such as cetyltrimethylammonium bromide (CTAB), bis(2-ethylhexyl)sodium sulfosuccinate (AOT), and the like.

The core solution 105 is encapsulated in a microcapsule 110 that is biologically benign. For example, when the enhanced chelator 100 is intended to be administered to a human patient, the biologically benign microcapsule 110 must be nontoxic to humans. In general, any suitable conventional biologically benign microcapsule may be utilized (e.g., poly(α-hydroxy acid) and liposome encapsulation systems). Poly(α-hydroxy acid) encapsulation systems include poly(D,L-lactic-co-glycolide) (PLGA) microspheres, poly(D,L-lactide) (DL-PLA) microspheres, and poly(L-lactide) (L-PLA) microspheres.

The core solution 105 is encapsulated within the microcapsule 110 using techniques known to those skilled in the art, such as an in situ polymerization method, a coacervation method, or an interfacial polymerization method. The use of the PLGA microcapsule in the exemplary embodiment of FIG. 1 is for purposes of illustration and not limitation. Other materials that may be suitable for the microspheres include, but are not limited to, DL-PLA, L-PLA, liposomes, urea-formaldehyde, vinylidene chloride-acrylonitrile copolymer, polyvinyl alcohol, polyvinyl butyral, polymethylmethacrylate, polyacrylonitrile, polyvinylidene chloride, polysulfone, and the like.

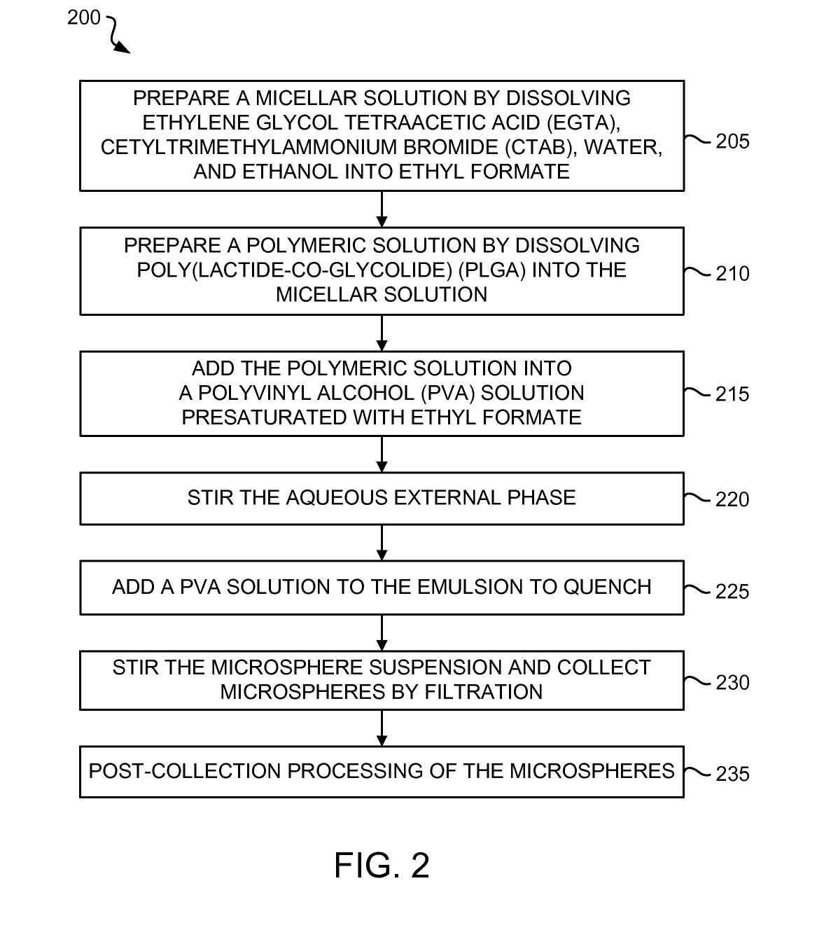

Preferably, the biologically benign microcapsule 110 is a PLGA microsphere prepared using a conventional reverse micellar microencapsulation technique that is modified to provide a core solution that includes a chelating agent and a volatile material. An example of a so-modified conventional reverse micellar microencapsulation technique is described below with reference to FIG. 2. Myriad conventional techniques useful for preparing PLGA microcapsules are well known. Exemplary conventional techniques suitable for preparing PLGA microcapsules include, but are not limited to, emulsion-based solvent evaporation, solvent extraction, spray drying, phase separation, coacervation, and interfacial polymerization. A reverse micelle-based encapsulation process and a methylene chloride-based double emulsion process for preparing tetracycline hydrochloride (TH) loaded PLGA microspheres are disclosed in H.-J. Kim et al., “Development of New Reverse Micellar Microencapsulation Technique to Load Water-Soluble Drug into PLGA Microspheres,” Archives of Pharmacal Research, Vol. 28, No. 3, pages 370-375, 2005, which is hereby incorporated herein by reference in its entirety.

FIG. 2 is a flow diagram illustrating an exemplary method 200 of fabricating an enhanced chelator in accordance with some embodiments of the present invention. In the method 200, the steps discussed below (steps 205-235) are performed. These steps are set forth in their preferred order. It must be understood, however, that the various steps may occur simultaneously or at other times relative to one another. Moreover, those skilled in the art will appreciate that one or more steps may be omitted. The materials used in the steps discussed below are commercially available.

The method 200 begins by preparing a micellar solution by adding 20 mg of EGTA, 30 mg of CTAB, 0.15 ml of anhydrous ethanol, and 0.15 ml of water into a vial containing 3 ml of ethyl formate (step 205). These materials are commercially available from suppliers such as Sigma-Aldrich Corp., St. Louis, Mo. The vial containing the micellar solution is then heated inside an oven at 30° C. for several hours. At least a portion of the EGTA and the ethanol in the micellar solution will ultimately provide a core solution of a microcapsule (e.g., corresponding to the core solution 105 of the microcapsule 110 shown in FIG. 1). The use of EGTA (as the “chelating agent”) and ethanol (as the “volatile material”) in the micellar solution of the exemplary method 200 is for purposes of illustration and not limitation. One skilled in the art will appreciate that one or more other suitable chelating agents and/or volatile materials may be used in lieu of, or in addition to, EGTA and/or ethanol.

Next, the method 200 continues by preparing a polymeric solution by dissolving 0.3 to 0.75 g of PLGA 75:25 (i.e., PLGA with a lactide:glycolide ratio of 75:25) into the micellar solution (step 210). The use of PLGA 75:25 in the polymeric solution of the exemplary method 200 is for purposes of illustration and not limitation. One skilled in the art will appreciate that one or more other suitable encapsulating materials may be used in lieu of, or in addition to, PLGA 75:25. PLGA 75:25 is commercially available from suppliers such as Birmingham Polymers, Inc., Birmingham, Ala.

The method 200 then continues by adding the polymeric solution into 20 ml of a 1% polyvinyl alcohol solution presaturated with ethyl formate (step 215). During this addition, the aqueous external phase is stirred at 475 rpm using a magnetic plate stirrer (step 220). After aqueous external phase is stirred for 5 minutes, an additional 60 ml of a 0.5% polyvinyl alcohol solution to added to the emulsion (step 225). Often referred to as the “quenching step”, the step 225 provides a quick extraction of the ethyl formate out of the polymeric phase into the aqueous external phase.

Next, the method 200 continues by stirring the microsphere suspension for 40 minutes and collecting the microspheres by filtration (step 230). The method 200 concludes with post-collection processing of the microspheres (step 235). This post-collection processing is performed using conventional techniques well known to those skilled in the art. For example, the microspheres collected in step 230 may be washed and dried, and then added to a conventional aqueous biological buffer solution. In an illustrative example, the microspheres collected in step 230 may be re-dispersed in 80 ml of a 0.5% polyvinyl alcohol solution and stirred for 1 hour, re-collected by filtration, dried under a vacuum for several hours, and then added to a conventional aqueous biological buffer solution.

A myriad of conventional biological buffers are commercially available from suppliers such as Sigma-Aldrich Corp., St. Louis, Mo. and AppliChem. Inc., New Haven, Conn. Conventional biological buffers include, for example, HEPES {N-(2-Hydroxyethyl)-piperazine-N□-ethanesulfonic acid}, MES {2-(N-Morpholino)-ethanesulfonic acid}, MOPS {3-(N-Morpholino)-propanesulfonic acid}, Tris {Tris(hydroxymethyl)-aminomethane}, BIS-Tris-Propane {1,3-Bis [tris(hydroxymethyl)-methylamino]propane}, etc. Conventional biological buffer solutions typically comprise of one or more conventional biological buffers with preservatives; salts such as NaCl, CaCl₂, and KCl; electrolytes such as Na⁺, K⁺, Ca²⁺, and Cl⁻; divalent metal ions such as Mg²⁺; nucleotides; proteins; and/or blood gas components such as CO₂ and O₂.

Unlike conventional chelators, the enhanced chelator will not react with components in aqueous biological buffer solutions, such as Tris buffer {Tris-(hydroxymethyl)-aminomethane}, KCl, divalent metal ions such as Mg²⁺, nucleotides, proteins, etc. Because the chelating agent is encapsulated in the microcapsule away from solution components with which the chelating agent would react, the enhanced chelator possesses significantly improved shelf-life even when stored in aqueous biological buffer solutions.

The enhanced chelator is activated at a predetermined elevated temperature defined by the boiling point of the core solution's volatile material. At this predetermined elevated temperature, the volatile material exerts a vapor pressure sufficient to rupture the microcapsule and thereby release the chelating agent from the microcapsule.

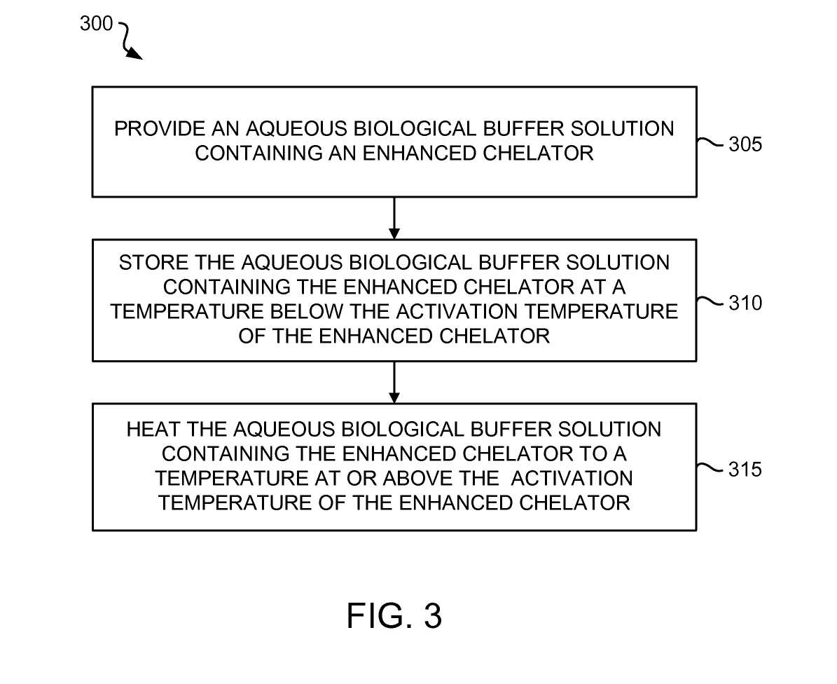

FIG. 3 is a flow diagram illustrating an exemplary method 300 of activating an enhanced chelator in accordance with some embodiments of the present invention. In the method 300, the steps discussed below (steps 305-315) are performed. These steps are set forth in their preferred order. It must be understood, however, that the various steps may occur simultaneously or at other times relative to one another. Moreover, those skilled in the art will appreciate that one or more steps may be omitted.

The method 300 begins by providing an aqueous biological buffer solution containing an enhanced chelator (step 305). The step 305 may, for example, correspond to performing the steps 205-235 discussed above with respect to the method 200 of FIG. 2. Alternatively, the step 305 may be performed by adding an enhanced chelator obtained from a supplier to an aqueous biological buffer solution. In another alternative, the step 305 may be performed by obtaining from a supplier an aqueous biological buffer solution that already contains an enhanced chelator.

The aqueous biological buffer solution containing the enhanced chelator is stored at a temperature below the activation temperature of the enhanced chelator (step 310). In other words, the aqueous biological buffer solution containing the enhanced chelator is stored at a temperature below the boiling point of the volatile material in the microcapsule's core solution. Hence, as long as the aqueous biological buffer solution containing the enhanced chelator is stored at an appropriate temperature below the activation temperature, the enhanced chelator remains in a non-active state. The enhanced chelator possesses significantly improved shelf-life in the aqueous biological buffer solution because the chelating agent in the microcapsule's core solution is separated from components of the aqueous biological buffer solution with which the chelating agent would react.

Activation of the enhanced chelator is achieved by heating the aqueous biological buffer solution containing the enhanced chelator to a temperature at or above the activation temperature of the enhanced chelator (step 315). This activation occurs at or above the boiling point of the volatile material (e.g., 80° C. in the case of ethanol) in the microcapsule's core solution. Hence, once the aqueous biological buffer solution containing the enhanced chelator is heated to this activation temperature, the enhanced chelator changes to and permanently remains in an active state. At the activation temperature, the volatile material in the microcapsule's core solution exerts a vapor pressure sufficient to rupture the microcapsule and thereby release the chelating agent from the microcapsule.

In the context of chelation therapy, subsequent to activation of the enhanced chelator in the aqueous biological buffer solution it may be necessary (depending on the activation temperature) to cool the solution containing the activated enhanced chelator so the solution can be safely administered. In an embodiment utilizing ethanol as the volatile material in the microcapsule's core solution, the aqueous biological buffer solution containing the activated enhanced chelator is cooled from the activation temperature (>80° C.) to a suitable temperature (e.g., room temperature) before administration to a patient.

One skilled in the art will appreciate that many variations are possible within the scope of the present invention. For example, although some embodiments of the present invention are described herein in the context of an enhanced chelator in an aqueous biological buffer solution to be administered to a human patient, the present invention may be utilized for other contexts. An enhanced chelator in accordance with some embodiments of the present invention may be used as an analytical tool (e.g., for the purpose of performing an assay), either in an aqueous biological buffer solution or some other solution. Also, an enhanced chelator in accordance with some embodiments of the present invention may be used for the purposes of recovery, purification and/or concentration. Thus, while the present invention has been particularly shown and described with reference to some embodiments thereof, it will be understood by those skilled in the art that these and other changes in form and detail may be made therein without departing from the spirit and scope of the present invention.

1. An enhanced chelator, comprising:
a chelating agent and a volatile material encapsulated in a biologically benign microcapsule, wherein the volatile material boils at a predetermined elevated temperature and exerts a vapor pressure at the predetermined elevated temperature sufficient to rupture the microcapsule and thereby release the chelating agent from the microcapsule.

2. The enhanced chelator as recited in claim 1, wherein the chelating agent is a manganese chelator.

3. The enhanced chelator as recited in claim 1, wherein the chelating agent is selected from a group of manganese chelators consisting of ethylene glycol tetraacetic acid (EGTA), para-aminosalicylic acid (PAS), 1,2-cyclohexylenedinitrilotetraacetic acid (CDTA), nitrilotriacetic acid (NAS), diethylenetriaminepentaacetic acid (DTPA), and combinations thereof.

4. The enhanced chelator as recited in claim 3, wherein the biologically benign microcapsule is a poly(lactic-co-glycolide) (PLGA) microsphere.

5. The enhanced chelator as recited in claim 4, wherein the volatile material is ethanol.

6. The enhanced chelator as recited in claim 1, wherein the biologically benign microcapsule is a poly(lactic-co-glycolide) (PLGA) microsphere.

7. The enhanced chelator as recited in claim 6, wherein the volatile material is ethanol.

8. An enhanced manganese chelator, comprising:
a chelating agent selected from a group of manganese chelators consisting of ethylene glycol tetraacetic acid (EGTA), para-aminosalicylic acid (PAS), 1,2-cyclohexylenedinitrilotetraacetic acid (CDTA), nitrilotriacetic acid (NAS), diethylenetriaminepentaacetic acid (DTPA), and combinations thereof;
a volatile material that boils at a predetermined elevated temperature;
a biologically benign microcapsule encapsulating the chelating agent and the volatile material, wherein volatile material exerts a vapor pressure at the predetermined elevated temperature sufficient to rupture the microcapsule and thereby release the chelating agent from the microcapsule.

9. The enhanced manganese chelator as recited in claim 8, wherein the biologically benign microcapsule is a poly(lactic-co-glycolide) (PLGA) microsphere.

10. The enhanced manganese chelator as recited in claim 9, wherein the volatile material is ethanol.

11. A method of preparing an enhanced chelator, the method comprising the steps of:
providing a solution comprising a chelating agent and a volatile material;
encapsulating the solution in a biologically benign microcapsule, thereby producing an enhanced chelator, comprising:
the chelating agent and the volatile material encapsulated in the biologically benign microcapsule, wherein the volatile material boils at a predetermined elevated temperature and exerts a vapor pressure at the predetermined elevated temperature sufficient to rupture the microcapsule and thereby release the chelating agent from the microcapsule.

12. The method as recited in claim 11, wherein the chelating agent is a manganese chelator.

13. The method as recited in claim 11, wherein the chelating agent is selected from a group of manganese chelators consisting of ethylene glycol tetraacetic acid (EGTA), para-aminosalicylic acid (PAS), 1,2-cyclohexylenedinitrilotetraacetic acid (CDTA), nitrilotriacetic acid (NAS), diethylenetriaminepentaacetic acid (DTPA), and combinations thereof.

14. The method as recited in claim 13, wherein the biologically benign microcapsule is a poly(lactic-co-glycolide) (PLGA) microsphere.

15. The method as recited in claim 14, wherein the volatile material is ethanol.

16. A method of activating an enhanced chelator, the method comprising the steps of:
providing an aqueous biological buffer solution containing an enhanced chelator comprising a chelating agent and a volatile material encapsulated in a biologically benign microcapsule, wherein the volatile material boils at a predetermined elevated temperature and exerts a vapor pressure at the predetermined elevated temperature sufficient to rupture the microcapsule and thereby release the chelating agent from the microcapsule;
heating the aqueous biological buffer solution to a temperature at or above the predetermined elevated temperature.

17. The method as recited in claim 16, wherein the step of providing an aqueous biological buffer solution comprises the step of storing the aqueous biological buffer solution at a temperature below the predetermined elevated temperature.

18. The method as recited in claim 16, wherein the chelating agent is a manganese chelator.

19. The method as recited in claim 16, wherein the chelating agent is selected from a group of manganese chelators consisting of ethylene glycol tetraacetic acid (EGTA), para-aminosalicylic acid (PAS), 1,2-cyclohexylenedinitrilotetraacetic acid (CDTA), nitrilotriacetic acid (NAS), diethylenetriaminepentaacetic acid (DTPA), and combinations thereof.

20. The method as recited in claim 19, wherein the biologically benign microcapsule is a poly(lactic-co-glycolide) (PLGA) microsphere.

21. The method as recited in claim 20, wherein the volatile material is ethanol.
